(19) United States
(12) Patent Application Publication
(10) Pub. No.: US 2016/0157784 A1
(43) Pub. Date: Jun. 9, 2016

(54) ELECTRONIC DEVICE

(71) Applicant: Kabushiki Kaisha Toshiba, Minato-ku (JP)

(72) Inventor: Shingo Suzuki, Sagamihara Kanagawa (JP)

(21) Appl. No.: 14/922,699

(22) Filed: Oct. 26, 2015

(30) Foreign Application Priority Data
Dec. 5, 2014 (JP) 2014-246556

(51) Int. Cl. A61B 5/00

(52) U.S. Cl. CPC A61B 5/7232; A61B 5/6801; A61B 5/0022; A61B 2560/0475

(57) ABSTRACT

According to one embodiment, an electronic device is configured to be worn by a user. The electronic device includes a sensor, a wireless transceiver, and a processor. The sensor measures vital data of the user. The wireless transceiver performs wireless communication with an external device. The processor selects a storage method of the vital data corresponding to strength of a signal transmitted from the external device and received by the wireless transceiver.

### CROSS-REFERENCE TO RELATED APPLICATIONS

This application is based upon and claims the benefit of priority from Japanese Patent Application No. 2014-246556, filed Dec. 5, 2014, the entire contents of which are incorporated herein by reference.

### FIELD

Embodiments described herein relate generally to an electronic device attachable to a user to measure vital data.

### BACKGROUND

In recent years, wearable devices having a sensing function have started to become widespread. Such devices, by being worn constantly, allow users to acquire their behavioral patterns and vital data. By referring to the behavioral patterns and vital data acquired through such a device, a user can check his or her daily activity and health.

For better performance in acquiring the behavioral patterns and vital data of the user, the device should be worn continuously for as long as possible; thus, a wearable device designed for extended use is demanded. In other words, there is a need to prolong the operating time of the wearable device to acquire a greater volume of data.

The prolongation of the data acquisition time of wearable device is demanded.

### BRIEF DESCRIPTION OF THE DRAWINGS

A general architecture that implements the various features of the embodiments will now be described with reference to the drawings. The drawings and the associated descriptions are provided to illustrate the embodiments and not to limit the scope of the invention.

FIG. 1 is an exemplary block diagram which shows an example of a schematic structure of a system including an electronic device of an embodiment.

FIG. 2 is an exemplary block diagram which shows an example of a circuit structure of the electronic device of the embodiment.

FIG. 3 is an exemplary block diagram which shows an example of a functional structure of a health care application program executed by the electrode device of the embodiment.

FIG. 4 shows a second-order difference scheme.

FIG. 5A, FIG. 5B, and FIG. 5C show examples of waveforms corresponding to sensing data, sensing data with first-order difference treatment, and sensing data with second-order difference treatment.

FIG. 6 is an exemplary histogram which shows frequency of amplitude of each of input signal, first-order difference signal, and second-order difference signal.

FIG. 7 is an exemplary view which shows an example of average code length with respect to heart rate.

FIG. 8 is an exemplary flowchart which shows an example of a process flow executed by a health care application program.

FIG. 9 is an exemplary flowchart which shows an example of a process flow executed by the health care application program.

FIG. 10 is an exemplary flowchart which shows an example of a process flow executed by the health care application program.

FIG. 11 is an exemplary flowchart which shows an example of a process flow executed by the health care application program.

### DETAILED DESCRIPTION

Various embodiments will be described hereinafter with reference to the accompanying drawings.

In general, according to one embodiment, an electronic device is configured to be worn by a user. The electronic device comprises a sensor, a wireless transceiver, and a processor. The sensor measures vital data of the user. The wireless transceiver performs wireless communication with an external device. The processor selects a storage method of the vital data corresponding to strength of a signal transmitted from the external device and received by the wireless transceiver.

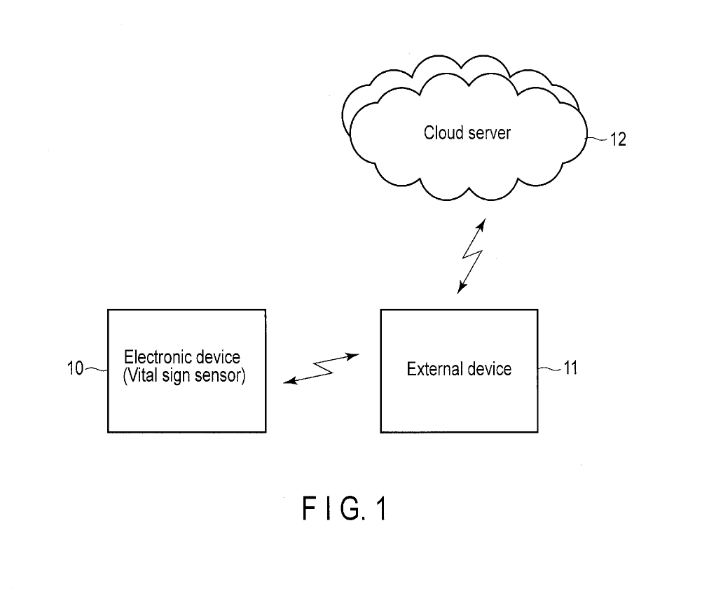

FIG. 1 is a block diagram which schematically shows a structural example of a system including an electronic device of one embodiment. Note that the electronic device in the embodiments is given as a vital sign sensor device. The vital sign sensor device 10 is a small, light, and thin device which is powered by a battery (for example, an in-device secondary battery). To measure vital data (sensing data) constantly, the vital sign sensor device 10 is adhered to the human body by, for example, adhesive tape or the like. Instead of such taping, the device may be attached to the body as a wrist band or an earpiece. The vital sign sensor device 10 acquires various items of vital data such as pulse, cardiogrphic, body temperature, and body motion data in synchronization and sends the vital data to an external device (such as a smartphone or PC) 11 wirelessly. Note that, the vital data may be stored temporarily in a flash memory inside the vital sign sensor device 10 before being sent to the external device. The vital sign sensor device 10 receives a control signal or the like from the external device 11 wirelessly. Note that the vital sign sensor device 10 may send identification data unique to the device 10 to the external device 11 together with the vital data. Furthermore, the vital sign sensor device 10 may send only one item of vital data selected from the various items of vital data to the external device 11. Or, the vital data to be acquired by the vital sign sensor device 10 may be focused on a single specific item of data. As explained later, if a condition indicative of low signal strength, low battery, or low memory is detected, the vital sign sensor device 10 may send warning data related to such a condition to the external device 11. Upon receipt of the vital data from the vital sign sensor device 10, the external device 11 may output the vital data on a display or may send the vital data to a cloud server 12. The cloud server 12 is a so-called external server.

The vital sign sensor device 10 includes a plurality of sensors to acquire various items of vital data at the same time. Analog front ends of the sensors are formed in different shapes to meet both requirements of high flexibility and high performance at the same time, and they are sometimes formed large. However, in the present embodiment, a plurality of analog front ends, CPU, and the like are all integrated on a single chip using a pseudo-system-on-a-chip (SoC) technique to achieve a sensor module of a few mm square. The pseudo-SoC technique is a technique related to component integration on a wafer, which achieves SoC-like miniaturization and SiP-like design freedom. The module with an antenna, battery, and a few peripheral components can be the vital sign sensor device 10 which is light (approximately 10 to 15 g), small, and thin (approximately a few mm). Note that the miniaturization can be achieved by an internal component substrate technique or dedicated LSI technique.

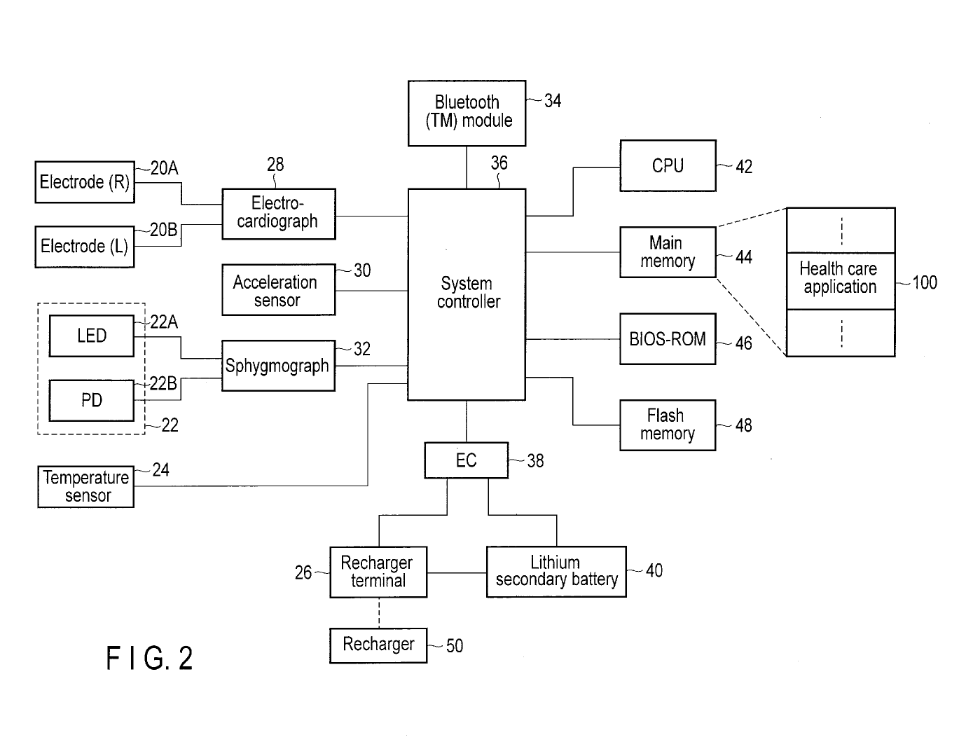

Now, with reference to FIG. 2, a circuit structure of the vital sign sensor device 10 is explained.

FIG. 2 is a block diagram which shows a circuit structure of the vital sign sensor device 10. The vital sign sensor device 10 includes, for example, cardiogram electrodes 20A and 20B, photoelectric unit 22, temperature sensor 24, recharger terminal 26, electrocardiograph 28, acceleration sensor 30, sphygmograph 32, Bluetooth™ module (wireless transceiver) 34, system controller 36, embedded controller (EC) 38, lithium secondary battery 40, CPU (processor) 42, main memory 44, BIOS-ROM 46, flash memory (nonvolatile memory) 48.

Cardiogram electrode (R) 20A and cardiogram electrode (L) 20B are connected to the electrocardiograph 28 used as an analog front end for cardiogram. The electrocardiograph 28 obtains the cardiogram by analyzing a time-series signal which is a sensing (sampling) result of an electronic potential between the cardiogram electrode (R) 20A and the cardiogram electrode (L) 20B. Furthermore, the electrocardiograph 28 acquires, from the cardiogram, an R-R interval (RRI) which is an interval between two R-waves corresponding to two consecutive heartbeats, and acquires a heart rate.

The photoelectric unit 22 is used to detect plethysmogram and includes a luminescent element (for example, a green LED) 22A which is a light source and a photodiode (PD) 22B which is a light receiver. A transparent window is provided in front of the photoelectric unit 22, and the light from the green LED 22A is irradiated upon a skin surface through the window, and then the reflection light is incident upon the PD 22B through the window. The green LED 22A and PD 22B are connected to the sphygmograph 32 which is an analog front end for pulse wave. The sphygmograph 32 detects a change in the reflection light which varies corresponding to a change in blood flow in capillary and analyzes the detected signal to acquire the pulse wave and pulse rate.

The temperature sensor 24, electrocardiograph 28, acceleration sensor 30, and sphygmograph 32 are connected to the system controller 36. The temperature sensor 24 measures a temperature on the body surface and the acceleration sensor 30 measures the body motion (for example, walking pace).

CPU 42 is a processor to control modules and components of the vital sign sensor device 10. As described above, the vital sign sensor device 10 analyzes the output from each sensor or a combination of outputs from sensors and continuously measures various vital data (for example, body temperature, skin temperature, pulse rate, heart rate, autonomic nerve activity index, blood pressure, blood oxygen concentration, walking pace, and sleeping hours).

Note that the blood pressure is derived from a pulse wave transit time (PWTT) based on a cardiogram wave peak (R-wave peak) and a pulse wave peak. The pulse wave transit time indicates a time gap between the appearance of R-wave in the cardiogram and the appearance of the terminal pulse wave. The pulse wave transit time is in inverse proportion to the blood pressure value. Thus, from the pulse wave transit time (PWTT), a change in the blood pressure can be acquired. Note that, in the measurement of the blood pressure, an initial value indicative of a relationship between the blood pressure value and the pulse wave transit time may be preset. For example, the blood pressure value of a user measured in an ordinary blood pressure measure and the pulse wave transit time in this measurement may be stored preliminarily as the initial value in the flash memory 48. Based on a change in the blood pressure derived from a current pulse wave transit time (PWTT) and the initial value (a relationship between the blood pressure value and the pulse wave transit time), a current blood pressure value of the user can be acquired. Or, instead of such an initial value based on the blood pressure value of a user measured in an ordinary blood pressure measure and the pulse wave transit time in this measurement, an average data set indicative of a relationship between the blood pressure value and the pulse wave transit time may be prepared to acquire a current blood pressure value of the user based on this average data set and a change in the blood pressure derived from a current pulse wave transit time (PWTT). Furthermore, the autonomic nerve activity index can be derived from a frequency analysis of the time series of the RRI. Furthermore, sleeping hours can be derived from Cole's formula, for example.

The system controller 36 is a bridge device which connects CPU42 to each module and component. A Bluetooth module 34, embedded controller (EC) 38, CPU42, main memory 44, BIOS-ROM 46, and flash memory 48 are connected to the system controller 36.

The embedded controller 38 is a power management controller which performs the power management of the vital sign sensor device 10 and controls, for example, electric charge of an in-device secondary battery such as lithium secondary battery 40. When the vital sign sensor device 10 is connected to a recharger 50, a recharger terminal 26 contacts a terminal of the recharger 50 and a recharging current from the recharger 50 is supplied to the vital sign sensor device 10 through the recharger terminal 26 for the recharge of the lithium secondary battery 40. The embedded controller 38 supplies the operation power to each module and each component using the power from the lithium secondary battery 40. The main memory 44 includes a health care application program 100, for example. The health care application program 100 is used to prolong the continuous service hour of the vital sign sensor device 10.

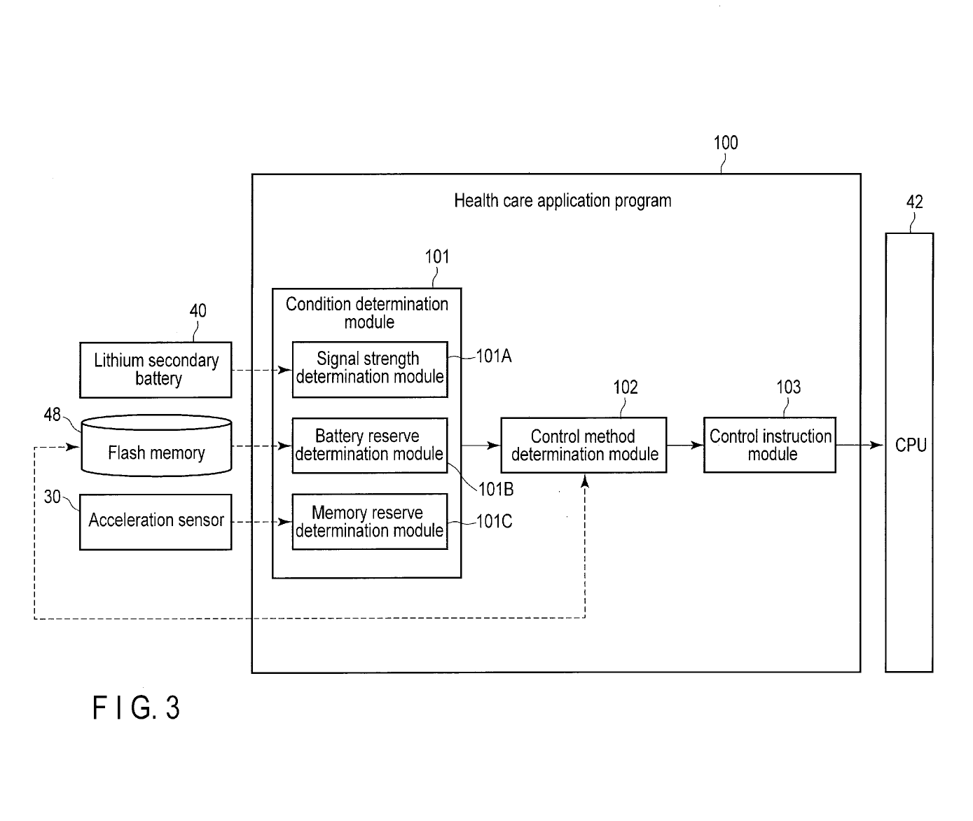

Here, the health care application program 100 is explained with reference to FIG. 3.

FIG. 3 is a block diagram which shows a functional structure of the health care application program 100. In FIG. 3, the health care application program 100 includes a condition determination module 101, control method determination module 102, and control instruction module 103. Hereinafter, the function of each of the units 101 to 103 is explained in detail.

The condition determination module 101 is a module configured to determine the current condition of the vital sign sensor device 10 (for example, data transfer in progress). As shown in FIG. 3, the condition determination module 101 further includes a signal strength determination module 101A, battery reserve determination module 101B, and memory reserve determination module 101C.

The signal strength determination module 101A recognizes the strength of the signal from the external device received at the Bluetooth module 34, determines whether or not the strength is greater than a predetermined threshold value, and sends the determination result to the control method determination module 102.

The battery reserve determination module 101B is a module configured to recognize the reserve of the lithium secondary battery 40 from its voltage and gas gauge (in other words, it is a module configured to obtain the data indicative of the reserve of the lithium secondary battery 40). The battery reserve determination module 101B recognizes the reserve of the lithium secondary battery 40 and determines whether or not the reserve is greater than a predetermined threshold value. If the determination indicates that the reserve is greater than the predetermined threshold value, the battery reserve determination module 101B informs the control method determination module 102 of the battery-good state. On the other hand, if the determination indicates that the reserve is less than or equal to the predetermined threshold value, the battery reserve determination module 101B informs the control method determination module 102 of the battery-low state. Note that the battery reserve determination module 101B reports the battery-good or battery-low state of the lithium secondary battery 40 in this embodiment; however, the battery reserve determination module 101B may simply inform the control method determination module 102 of the exact reserve of the lithium secondary battery 40, that is, the specific battery charge level.

The memory reserve determination module 101C is a module configured to recognize the available capacity of the flash memory 48 (in other words, it is a module configured to obtain the data indicative of available capacity of the flash memory 48). The memory reserve determination module 101C recognizes the available capacity of the flash memory 48 and determines whether or not the available capacity is greater than a predetermined threshold value. If the determination indicates that the available memory is greater than the predetermined threshold value, the memory reserve determination module 101C informs the control method determination module 102 of the memory-good state. On the other hand, if the determination indicates that the available capacity is less than or equal to the predetermined threshold value, the memory reserve determination module 101C informs the control method determination module 102 of the memory-low state. Note that the memory reserve determination module 101C reports the memory-good or memory-low state of the flash memory 48 in this embodiment; however, the memory reserve determination module 101C may simply inform the control method determination module 102 of the exact available capacity of the flash memory 48.

Note that, although it is not shown in FIG. 3, the condition determination module 101 may further include a behavior determination module in addition to the above determination modules 101A, 101B, and 101C. The behavior determination module recognizes, for example, a walking pace of the user measured by an acceleration sensor 32, and determines whether or not the walking pace is greater than a predetermined threshold value. Based on a measurement result, the behavior determination module informs the control method determination module 102 of the behavior of the user, in other words, a walking motion or a running motion of the user. If the behavior determination module is added to the condition determination module 101, the control method of the vital sign sensor device 10 can be determined more accurately.

The control method determination module 102 is a module to determine how to control the vital sign sensor device 10 based on various data informed from the condition determination module 101. Specifically, the control method determination module 102 as a selection means selects a data storage method based on the vital data.

The storage method includes items to determine what method is used to compress the vital data measured by the vital sign sensor device 10, what sensing interval is used for the measurement, what resolution is used to resolve the vital data (that is, what bit digital data is used for the vital data), and what storage location is used based on the vital data. As the data storage location based on the vital data, the external device 11 or the flash memory 48 may be adopted, for example.

If the wireless communication is poor, the power consumption of the Bluetooth module 34 may probably increase and packet loss may probably occur. Consequently, the lithium secondary battery 40 is consumed largely and a time available to obtain the vital data is shortened. Therefore, the control method determination module 102 selects a storage method of the vital data based on the signal strength from the external device 11 received by the Bluetooth module 34. If a storage method suitable for the vital data is selected based on the signal strength, the power consumption can be suppressed and data loss due to the communication error can be prevented.

The storage method includes the following modes 1 to 5, for example. Now, modes 1 to 5 are explained one by one.

[Mode 1: Lossless Transmission]

In mode 1, a second-order difference compression scheme which is a lossless compression scheme is used. The sensing data acquired are compressed without a loss, the compressed sensing data are transmitted to the external device 11 in real time, and the sensing data are stored in the external device 11. In the present embodiment, the second-order difference scheme is adopted as the lossless compression scheme. However, the lossless compression scheme may be, instead of the second-order difference scheme, an input digital signal itself, or a linear prediction scheme, or the like.

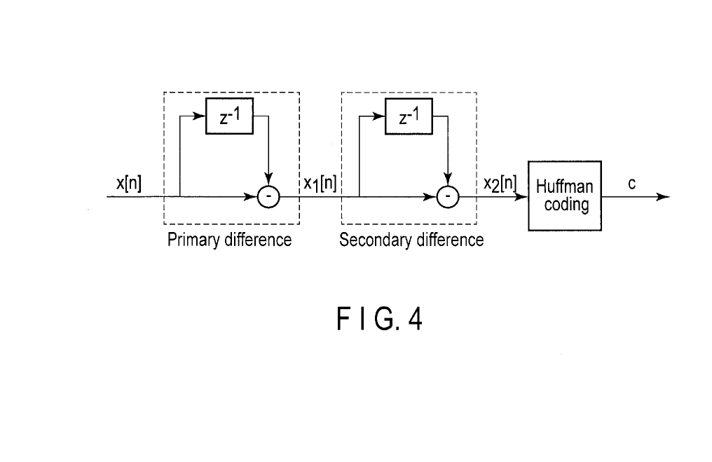

FIG. 4 shows the second-order difference scheme. First-order difference value x₁[n] is derived from a difference between sensing data (input signal) x[n] and x[n−1] which is x[n] with a time delay z⁻¹. Then, second-order difference value x₂[n] is derived from a difference between first-order difference value x₁[n] and x[n−1] which is x₁[n] with a time delay z⁻¹. A value c which is compressed second-order difference value x₂[n] is derived from Huffman coding. If the second-order difference is applied to the sensing data, a difference signal generated therefrom shows strong bias and consequently, the compression rate is increased.

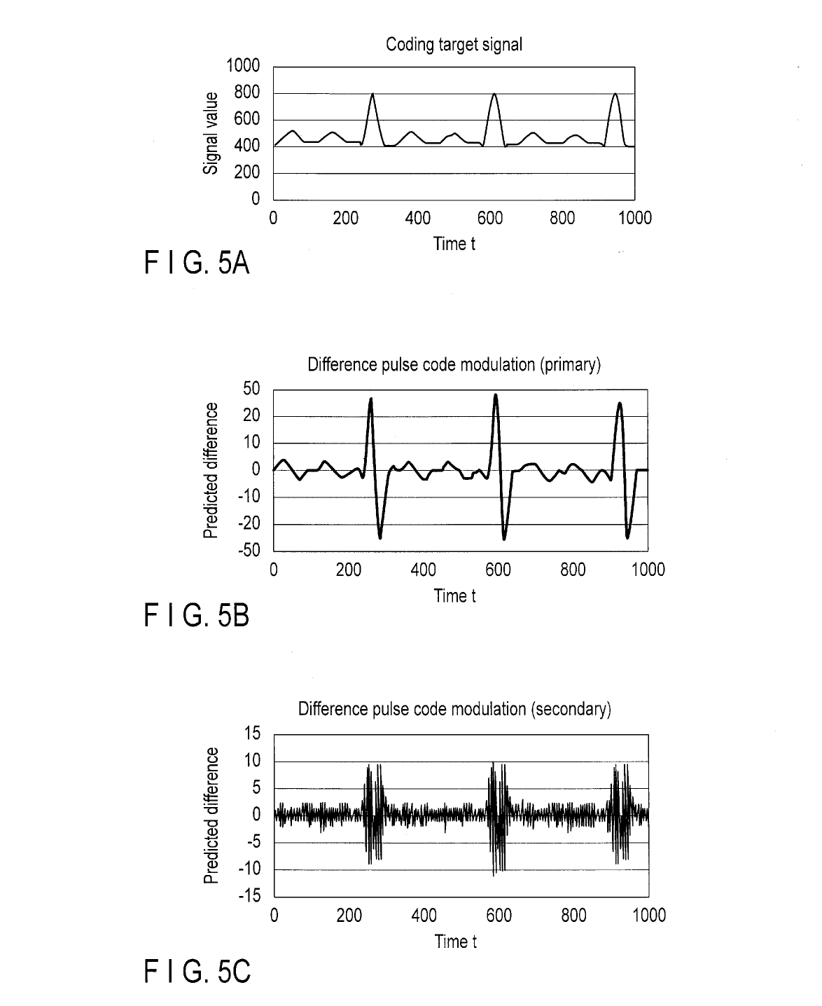

FIG. 5(A) shows a waveform of the sensing data (input signal). FIG. 5(B) is a waveform of the sensing data (input signal) subjected to the first-order difference. FIG. 5(C) is a waveform of the sensing data (input signal) subjected to the second-order difference.

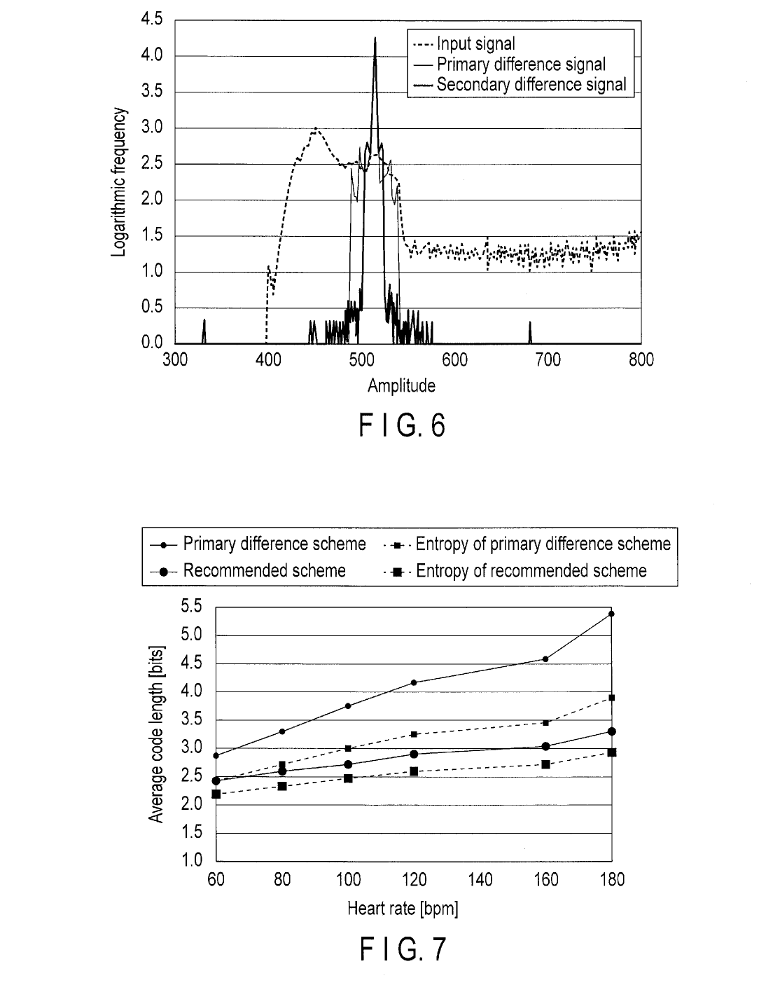

FIG. 6 shows a histogram indicating frequency of amplitude of input signal, first-order difference signal, and second-order difference signal. As shown in FIG. 6, the second-order difference signal has its peak when the amplitude is approximately 500.

FIG. 7 shows an average code length with respect to heart rate (sensing data). FIG. 7 shows the first-order difference scheme, second-order difference scheme, entropy of the first-order difference scheme, and entropy of the second-order difference scheme. As can be understood form FIG. 7, the compression rate of the second-order difference scheme is greater than any other schemes. Furthermore, a deviation due to the heart rate is kept low and a good result is obtained.

[Mode 2: Lossless Transmission]

In mode 2, the second-order difference compression scheme which is a lossless compression scheme is used as in mode 1. The sensing data acquired are compressed without a loss, the compressed sensing data are transmitted to the external device 11 in real time, and the sensing data are stored in the external device 11. However, in mode 2, parameters of the data subjected to sensing are prepared roughly as compared to the lossless transmission of mode 1, and thus, the data size based on the vital data is prepared less than the data size in mode 1. As a compression scheme, the second-order difference scheme is adopted in mode 2 as in mode 1. For example, the sampling rate as the parameters may be extended, or the quantization bit number of the amplitude as the parameters may be reduced.

[Mode 3: Lossy Transmission and Only High Frequency Component Storage]

Mode 2 with a different compression scheme is used in this mode. In mode 3, a compression scheme is a lossy compression scheme. The data size to be stored in mode 3 is less than that of mode 2. The compression scheme may be wavelet transformation which is a high frequency band process, or may be the second-order difference scheme with a quantization process. If the wavelet transformation is adopted, the high frequency components may be stored in the memory. If the second-order difference scheme is adopted, a difference acquired in the quantization process of the second-order difference result may be stored in the memory.

[Mode 4: Lossless Storage]

In mode 4, the compression scheme of the second-order difference is used to compress acquired sensing data without a loss and to store the compressed sensing data in the flash memory 48.

[Mode 5: Lossless Storage]

As compared to the lossless storage of mode 4, in mode 5, parameters of the data subjected to sensing (sampling rate or quantization bit number of amplitude) are prepared roughly to reduce the data size, and the data based on the vital data are stored in the flash memory 48. As the compression scheme, the second-order difference scheme is adopted.

Note that if the memory has limited capacity, a storage version of mode 3 may be adopted. In that case, the data to be stored correspond to the data to be transmitted in the lossy transmission scheme.

Now, returning back to the explanation of FIG. 3, the control instruction module 103 outputs instructions for the control of modules and components to CPU 42 based on the storage scheme selected by the control method determination module 102. CPU42 receives the instructions output from the control instruction module 103 and performs the control based on the instructions.

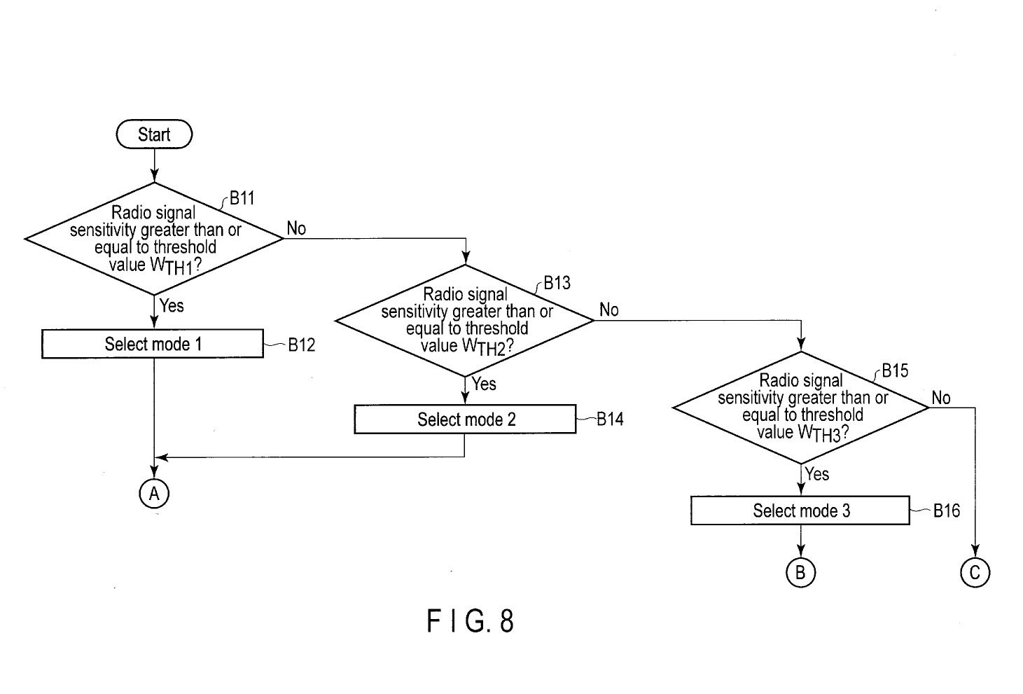

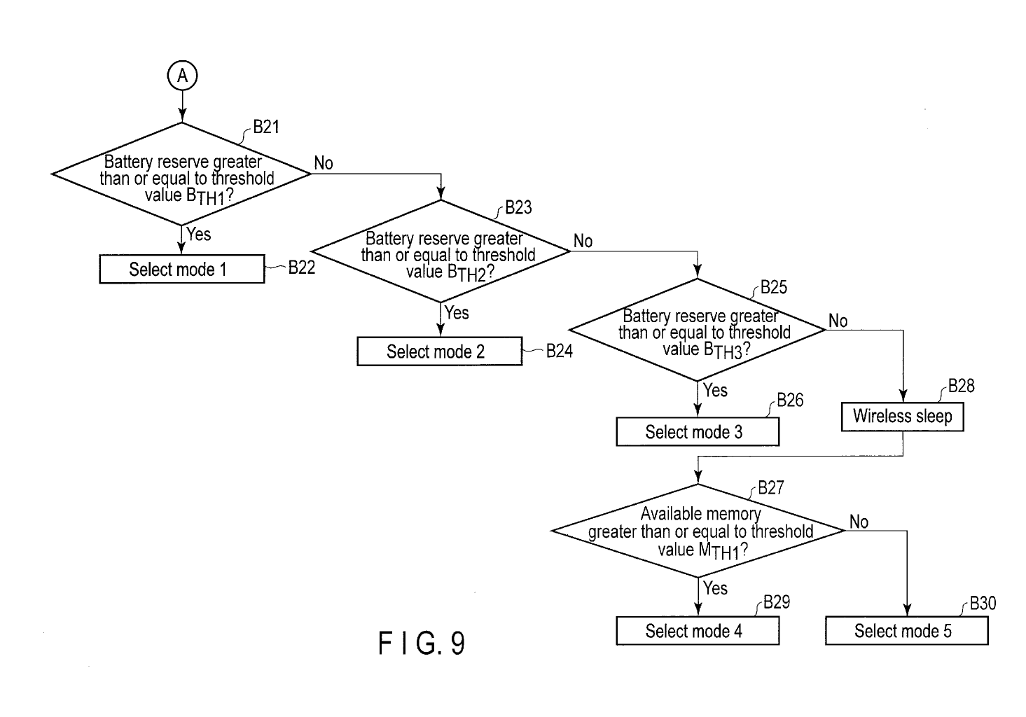

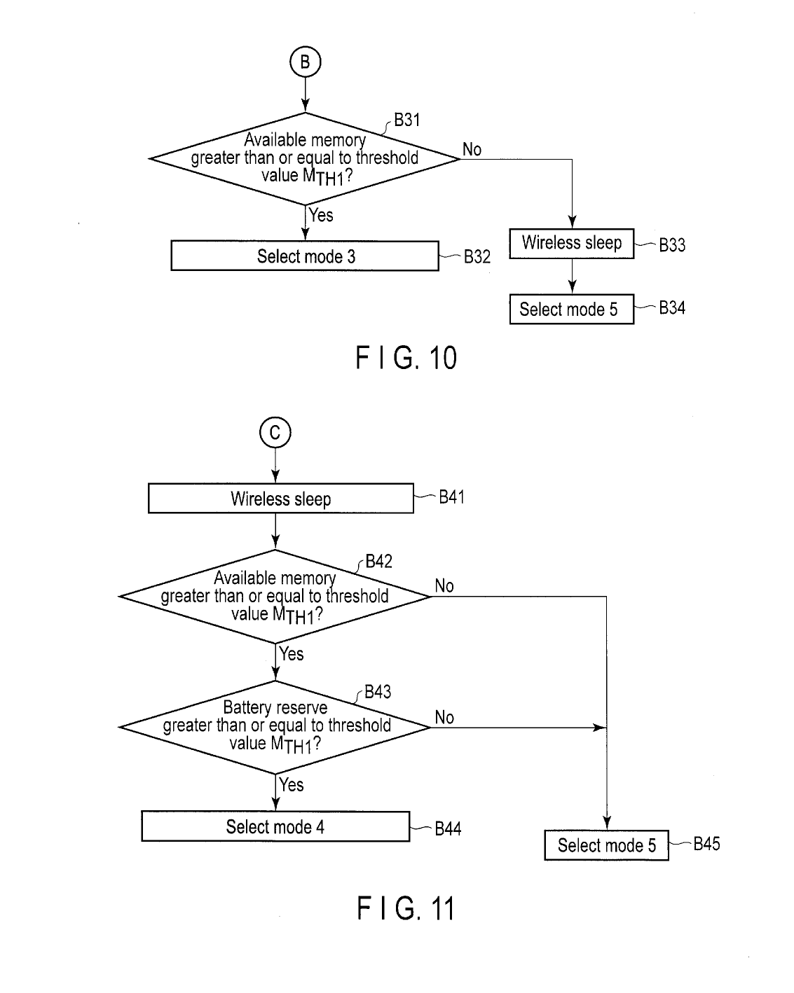

Now, with reference to the flowcharts of FIGS. 8 to 11, an example of a process executed by the health care application program 100 is explained.

Initially, a determination based on radio signal sensitivity is performed. The signal strength determination module 101A determines whether the radio signal sensitivity is greater than or equal to a threshold value W_{TH1} (block B11). If the radio signal sensitivity is greater than or equal to threshold value W_{TH1} (yes in block B11), the control method determination module 102 selects a lossless transmission (mode 1) as the data transmission mode (block B12). Mode 1 is the second-order difference scheme which is tolerant of a change in signal components.

The battery reserve determination module 101B determines whether or not the battery reserve is greater than or equal to a threshold value B_{TH1} (block B12). If the battery reserve is greater than or equal to threshold value B_{TH1} (Yes in block B21), the control method determination module 102 selects the lossless transmission (mode 1) as the data transmission mode (block B22). If the battery reserve is less than threshold value BTH1 (No in block B21), the battery reserve determination module 101B determines whether or not the battery reserve is greater than or equal to a threshold value B_{TH2} (block B23). If the battery reserve is greater than or equal to threshold value B_{TH2} (Yes in block B23), the control method determination module 102 selects a lossless transmission (mode 2) as the data transmission mode (block B24). If the battery reserve is less than threshold value B_{TH2}, the battery reserve determination module 101B determines whether or not the battery reserve is greater than or equal to a threshold value B_{TH3} (block B25). If the battery reserve is greater than or equal to a threshold value B_{TH3} (Yes in block B25), the control method determination module 102 selects a lossless transmission (mode 3) as the data transmission mode (block B26). If the battery reserve is less than threshold value B_{TH3} (No in block B25), the control instruction module 103 switches the Bluetooth module 34 to sleep mode (block B27).

The memory reserve determination module 101C determines whether or not the available capacity in the flash memory 48 is greater than or equal to a threshold value M_{TH1} (block B28). If the available capacity is greater than or equal to threshold value M_{TH1} (Yes in block B28), the control method determination module 102 selects a lossless storage (mode 4) as the data storage mode (block B29). If the available capacity is less than threshold value M_{TH1} (No in block B28), the control instruction module 103 selects a lossy storage (mode 5) as the data storage mode (block B30).

If the radio signal sensitivity is less than threshold value W_{TH1} (No in block B11) in block B11, the signal strength determination module 101A performs redetermination. In this redetermination, the signal strength determination module 101A determines whether or not the radio signal sensitivity is greater than or equal to a threshold value W_{TH2} (block B13). If the radio signal sensitivity is greater than or equal to threshold value W_{TH2} (Yes in block B13), the control method determination module 102 selects the lossless transmission (mode 2) as the data transmission mode (block B14). In mode 2, parameters used in sensing (sampling rate and quantization) are changed. On the other hand, if the radio signal sensitivity is less than threshold value W_{TH2} (No in block B13), the signal strength determination module 101A determines whether or not the radio signal sensitivity is greater than or equal to a threshold value W_{TH3} (block B15). If the radio signal sensitivity is greater than or equal to threshold value W_{TH3} (Yes in block B15), the control method determination module 102 selects the lossy transmission and high frequency component storage (mode 3) as the data transmission mode (block B16).

The memory reserve determination module 101C determines whether or not the available capacity of the flash memory 48 is greater than or equal to threshold value M_{TH1} (block B31). If the available capacity is greater than or equal to threshold value M_{TH1} (Yes in block B31), the control method determination module 102 selects the lossless storage (mode 4) as the data storage mode (block B32). If the available capacity is less than threshold value M_{TH1} (No in block B31), the control instruction module 103 switches the Bluetooth module 34 to sleep mode (block B33). The control instruction module 103 selects the lossy storage (mode 5) as the data storage mode (block B34).

If the radio signal sensitivity is less than threshold value W_{TH3} in block B15 (No in block B15), the memory reserve determination module 101C performs the determination by the available capacity in the flash memory 48. In the following flow, a scheme which does not use a wireless device is adopted. Thus, the control instruction module 103 switches the Bluetooth module 34 to sleep mode (block B41). Then, a comparison based on the available capacity of the flash memory 48 is performed.

The memory reserve determination module 101C determines whether or not the available capacity of the flash memory 48 is greater than or equal to threshold value M_{TH1} (block B42). If the available capacity is greater than or equal to threshold value M_{TH1} (Yes in block B42), the battery reserve determination module 101E determines whether or not the battery reserve is greater than or equal to threshold value B_{TH1} (block B43). If the battery reserve is greater than or equal to threshold value B_{TH1} (Yes in block B43), the control method determination module 102 selects the lossless storage (mode 4) as the data storage mode (block B44).

If the available capacity is less than threshold value M_{TH1} (No in block B31), or if the battery reserve is less than threshold value B_{TH1} (No in block B43), the control instruction module 103 selects the lossy storage (mode 5) as the data storage mode (block B45)

Note that, if conditions such as radio signal strength, battery reserve, and available capacity of the memory are improved during the storage process in the flash memory 48, the communication speed is changed to adapt to the amount of data stored in the flash memory 48 to reduce data upload time.

Furthermore, if the Bluetooth module 34 is switched to sleep mode, a notice of the switch may be sent to the external device 11 beforehand. If the user decides to maintain the communication state, the Bluetooth module 34 is not switched to sleep mode and only a notice of poor radio condition may be sent to the external device 11.

The storage method may be determined based on motion data such as acceleration rate or the like using the same scheme as for the radio signal sensitivity. For example, if the time-average motion is great, that is, if an intensive exercise such as running is being performed, body motion, heartbeat, and pulse rate tend to be greater and faster. Thus, the sampling interval is set to shorter to grasp changes during the exercise in detail.

On the other hand, if the time-average motion is small, heartbeat and pulse rate tend to be apparently calm. Thus, the sampling interval is set longer to suppress unnecessary data storage.

As can be understood from the above, the present embodiment selects the storage method of the vital data based on the strength of the signal transmitted from the external device 11 and received by the Bluetooth module 34, and thus, the time used to acquire the vital data can be prolonged.

Note that the process in the present embodiment can be achieved by a computer program. Thus, if such a computer program is installed and executed in a computer through a computer-readable recording medium which stores the computer program, the same advantage obtained by the present embodiment can easily be achieved.

The various modules of the systems described herein can be implemented as software applications, hardware and/or software modules, or components on one or more computers, such as servers. While the various modules are illustrated separately, they may share some or all of the same underlying logic or code.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic device configured to be worn by a user, the device comprising:
a sensor configured to measure vital data of the user;
a wireless transceiver configured to perform wireless communication with an external device; and
a processor configured to select a storage method of the vital data corresponding to strength of a signal transmitted from the external device and received by the wireless transceiver.

2. The electronic device of claim 1, wherein the processor is configured to select, when the strength is greater than or equal to a first threshold value, a first storage method which transmits first data based on the vital data to the external device.

3. The electronic device of claim 2, wherein the processor is configured to select, when the strength is less than the first threshold value and is greater than or equal to a second threshold value, a second storage method which transmits second data to the external device, wherein size of the second data based on the vital data is less than the first data.

4. The electronic device of claim 3, wherein the first data and the second data are compressed in a lossless compression scheme.

5. The electronic device of claim 4, wherein the lossless compression scheme is a second-order difference scheme.

6. The electronic device of claim 3, wherein the processor selects, when the strength is less than the second threshold value and is greater than or equal to the a third threshold value, a third storage method which transmits third data to the external device, wherein size of the third data based on the vital data is less than the second data.

7. The electronic device of claim 6, wherein the third data is compressed by lossy compression scheme.

8. The electronic device of claim 6, further comprising a nonvolatile memory, wherein the processor is configured to select, when the strength is less than the third threshold value, a fourth storage method which stores fourth data based on the vital data in the nonvolatile memory.

9. A control method for an electronic device configured to be wearable by a user and comprising a sensor configured to measure vital data of the user and a wireless transceiver configured to perform wireless communication with an external device, the method comprising:
measuring the vital data of the user by the measurement module; and
selecting a storage method of the vital data based on strength of a signal transmitted from the external device and received by the wireless transceiver.

10. A computer readable, non transitory storage medium configured to store a computer program which is executable by a computer configured to be wearable by a user and comprises a sensor configured to measure vital data of the user and a wireless transceiver configured to perform wireless communication with an external device, the computer program controlling the computer to execute function of:
selecting a storage method of vital data based on strength of a signal transmitted from the external device and received by the wireless transceiver.
